(19) United States
(12) Patent Application Publication
(10) Pub. No.: US 2019/0183744 A1
(43) Pub. Date: Jun. 20, 2019

(54) METHODS OF MAKING COLORED WET WIPES, APPARATUSES THEREFOR, AND PRODUCTS

(71) Applicant: ALBAAD MASSUOT YITZHAK LTD., MASSUOT YITZHAK (IL)

(72) Inventors: Chanan ADLER, Jerusalem (IL); Keren SHABTAI, Mazkeret Batya (IL); Ifat HAMMER, Rehovot (IL)

(73) Assignee: ALBAAD MASSUOT YITZHAK LTD., MASSUOT YITZHAK (IL)

(21) Appl. No.: 16/221,744

(22) Filed: Dec. 17, 2018

Related U.S. Application Data
(60) Provisional application No. 62/599,723, filed on Dec. 17, 2017.

(51) Int. Cl. A61K 8/02; D04H 1/64

(52) U.S. Cl. CPC A61K 8/0208; C11D 17/049; D04H 1/64; A61K 8/0241

(57) ABSTRACT

Disclosed are systems and methods for manufacturing colored wet wipes that avoid assembling colored fibers. The methods comprise preparing nonwovens from non-colored fibers, wetting the nonwovens with a functional liquid and a colorant (together or separately) to produce colored wet wipes, and packaging the wet wipes in a wet state.

### CROSS REFERENCE TO RELATED APPLICATIONS

This application claims the benefit of U.S. Provisional Patent Application No. 62/599,723 filed on Dec. 17, 2017, which is incorporated herein by reference in its entirety.

### FIELD OF THE INVENTION

The invention is directed to methods of producing colored wipes and especially “wet wipes”, apparatuses therefor and products.

### BACKGROUND OF THE INVENTION

“Wet wipes” are sheets of fabric, often made of nonwoven, disposable fiber sheets packaged wet and provided to the consumer in a wet state.

The conventional method for producing colored wipes uses colored fibers. The colored fibers are “assembled” to form the fabric, thus obtaining a colored fabric. The fabric is then wetted to produce colored wet wipes.

### SUMMARY OF THE INVENTION

The following is a simplified summary providing an initial understanding of the invention. The summary does not necessarily identify key elements nor limit the scope of the invention, but merely serves as an introduction to the following description.

One aspect of the invention provides a system for manufacturing colored wet wipes, the system comprising: a preparing unit configured to prepare nonwovens from non-colored fibers;

at least a first liquid supply reservoir; at least one wetting unit configured to apply at least a first liquid formulation from the first liquid supply reservoir to the nonwovens; and a packaging unit adapted to wrap the wet wipe in a package in a wet state; wherein the first liquid formulation comprises a colorant; and wherein a functional liquid adapted to wet the nonwovens and maintain the colored wet wipes wet in the package is included in the first liquid formulation or applied separately.

In one embodiment the at least one wetting unit comprises at least one first sub-unit configured to apply a liquid colorant in the first liquid formulation and color the nonwovens with the liquid colorant to produce colored nonwovens, and a least one second sub-unit configured to apply a second liquid formulation comprising a functional liquid and wet the colored nonwovens with the functional liquid to produce colored wet wipes.

In one embodiment the system further comprises a drier configured to dry the colored nonwovens intermediate the first sub-unit applying the liquid colorant and the second sub-unit wetting with the functional liquid.

In one embodiment the preparing unit is a spunlace unit configured to prepare spunlace nonwovens by hydroentanglement.

In one embodiment the first liquid formulation comprises a colorant and a functional liquid.

Another aspect of the invention provides a method for manufacturing colored wet wipes, the method comprising: preparing nonwovens from non-colored fibers; wetting the non-colored nonwovens with a liquid formulation comprising a colorant to form colored nonwovens; wetting the colored nonwovens with a functional liquid adapted to maintain the wet wipes in a wet state to form colored wet wipes; and packaging the colored wet wipes in a wet state.

In one embodiment the method further comprises wetting the non-colored nonwovens with the colorant and the functional liquid simultaneously in a single wetting step.

In one embodiment the method further comprises wetting non-colored nonwovens with the liquid composition comprising a colorant and thereafter wetting the colored nonwovens with the functional liquid to form a wet wipe.

In one embodiment the method further comprises drying the colored nonwovens prior to wetting with the functional liquid.

In one embodiment the method further comprises preparing the nonwovens by hydroentanglement to obtain spunlace nonwovens.

Another aspect of the invention provides wet wipes colored with a colorant, wherein the colorant is configured to adhere to the nonwovens; and wherein the colorant comprises an extract of: a vegetable, an animal, a fungus, an insect, bacteria, a mineral, a plant, or algae or a combination thereof.

These, additional, and/or other aspects and/or advantages of the present invention are set forth in the detailed description that follows; possibly inferable from the detailed description; and/or learnable by practice of the present invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a prior art process of dying wet wipes.

FIGS. 2A and 2B are schematic illustration of processes of dying wet wipes according to some embodiments of the invention.

FIG. 3A is a schematic illustration of a process of preparing nonwovens according to some embodiments of the invention.

FIG. 3B is a schematic illustration of preparing interfolded wet wipes according to some embodiments of the invention.

FIG. 3C is a schematic illustration of an apparatus for preparing and coloring nonwovens according to some embodiments of the invention.

FIG. 4 is a flow chart illustrating exemplary methods according to some embodiments of the invention.

It will be appreciated that for simplicity and clarity of illustration, elements shown in the figures are schematic and have not necessarily been drawn to scale.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

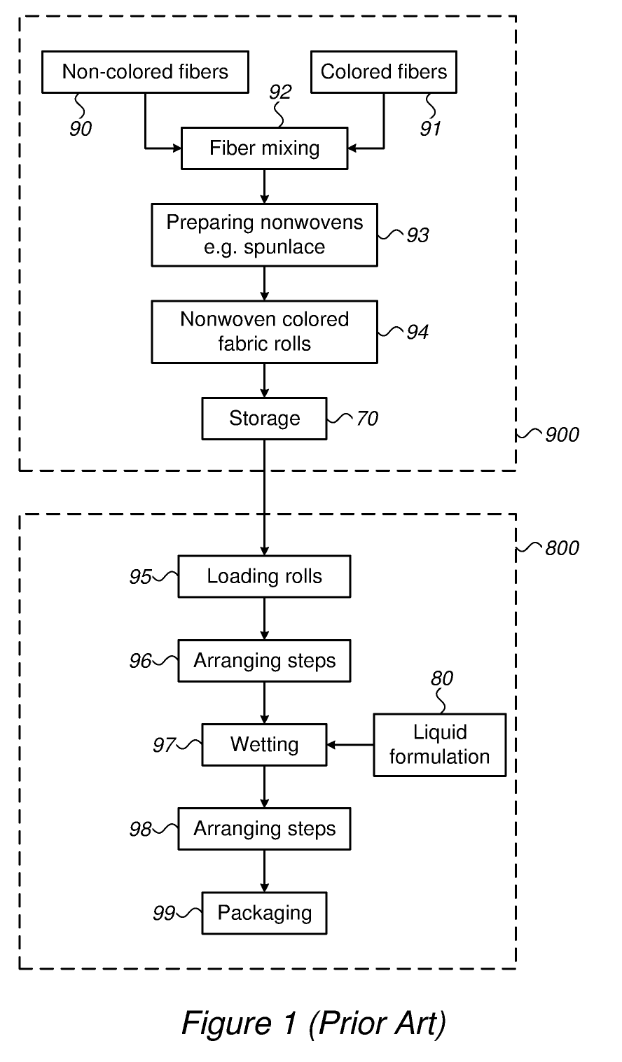

FIG. 1 is a scheme of a prior art process of preparing colored wet wipes. As discussed above, usually in the wet wipe industry, colored wet wipe fabrics, also known as ‘nonwovens’, are produced by mixing 92 colored fibers 91 and optionally non-colored fibers 90, at the desired ratio, to form the colored fabric. The coloring is done during the process of producing the fabric by “assembling” colored fibers. The “assembling” is described in detail below.

Nonwoven fabric is a fabric made from fibers or filaments from vegetarian or synthetic sources of different sizes bonded together by chemical, mechanical, heat or solvent treatment and which are neither woven nor knitted. The formation of the fabric can be made in a dry or wet environment. The fiber bonding can be done mechanically (needles, or water jet) or by thermobonding (pressure and heating), alternatively chemicals may be added as binders. Methods of preparing of nonwoven sheets include, without limitation, hydroentanglement, referred to as spunlace, needlepunching, airlaid and wetlaid techniques. In some embodiments, the wet wipes according to the invention are prepared from spunlace nonwoven fabric.

The present invention provides in one embodiment, a process comprising coloring a white fabric (or fabric of any other color), i.e. nonwovens, to a desired color after the fabric is already assembled/produced. The novel process of this invention comprise coloring the fabric using a colored liquid that wets and colors the white fabric (or fabric of any other color). The coloring of the fabric is done in the converting process as apposed to the assembling process of the prior art. Other embodiments of this process include coloring the fabric using a colored fluid, e.g. gas/aerosol or a gas/liquid mixture.

Wet wipes of this invention may be used for many applications in many different areas. For example, wet wipes of this invention may be used for personal care, such as beauty products and hygiene product, for household upkeep, such as cleaning and polishing, in hospitals, such as using the wipes for disinfecting or pain relief, in clinics and in care centers, in offices, in factories, such as industrial cleaning wipes, for pet care, such as eye, ear, or dental cleaning wipes, in auto-repair centers, for military uses etc.

FIG. 1 illustrates the prior art process of preparing wet wipes. The first section 900 illustrates the assembly process of preparing colored nonwoven rolls. In this section non-colored fibers 90 (such as white or other natural color fibers) are mixed 92 with colored fibers 91 at the appropriate ratio to produce the desired color. As used herein, non-colored means having a starting color other than the final color of the wet wipe. For example, some fibers are bleached and treated with TiO2 pigment before processing, but these are still considered non-colored fibers.

The fibers are then processed to produce nonwovens 93, for example by the spunlace process. The nonwovens are wound up into rolls 94 and placed in storage 70.

The second section 800 of FIG. 1 illustrates the converting process of wetting nonwovens to produce wet wipes. The rolls from section 900 are loaded in to a machine unit 95 and arranged 96. The arranging steps may include slitting and cutting the fabric into an intermediate width or to the desired wet wipe size. The arranging steps may include, in addition or instead of the cutting/slitting, folding the fabric or other arranging steps such as stacking, layering, pressing etc. The fabric is then wetted 97 by a liquid formulation 80. The liquid formulation may be water or a functional liquid(s) or any combination thereof. The wetted fabric then passes through additional arranging steps 98 which may include cutting to the desired wet wipe size if that was not done in the first set of arranging steps 96. The arranging steps may include, in addition or instead of the cutting, folding the fabric or other arranging steps such as stacking, layering, pressing etc. The wet wipes are then packaged 99. The wipes may be packed one by one (sachets), i.e. each wipe separately, such as used in restaurants and airlines, or multiple wipes in a package (flowpack), such as used for baby wipes or cleaning wipes. A packaging unit is adapted to wrap the wet wipe in a package in a wet state, often sealed in an envelope of low moisture permeability, such as foil or thin plastic film. Low moisture permeability packaging and packaging units are known in the art.

“Converting process” refers to the process by which the wet wipes are formed and/or processed. The converting process may include slitting, cutting, wetting, coloring and/or folding. Following the converting process other processes such as packaging or pre-packaging processing can be applied. The converting process may include slitting a web material or cutting sheets to an intermediate with and/or cutting to the final size of the wipes. The converting process may include other treatments such as coating the fabric etc. In some embodiments, a converting line is the process line or production line on which the converting process is conducted. A converting machine can be part of a production line as known in the art.

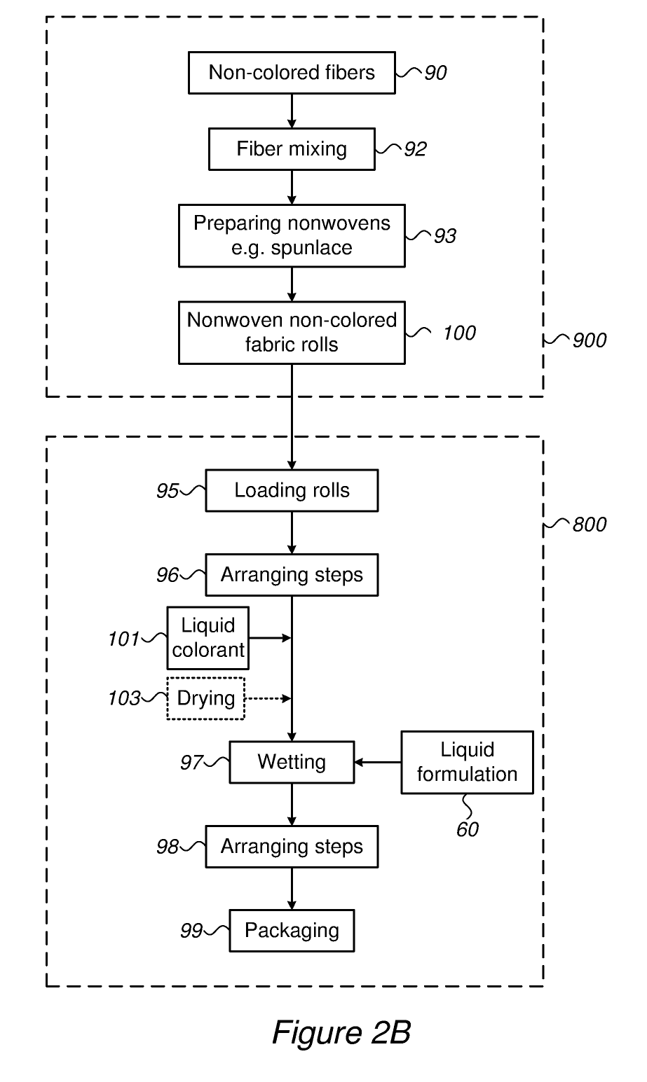

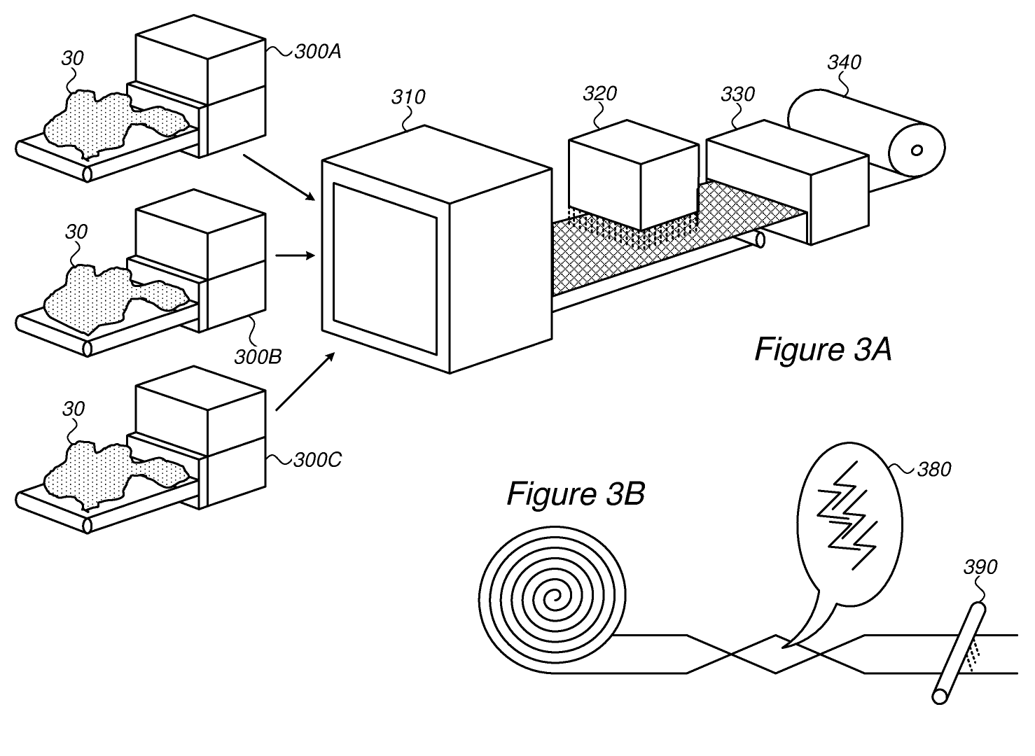

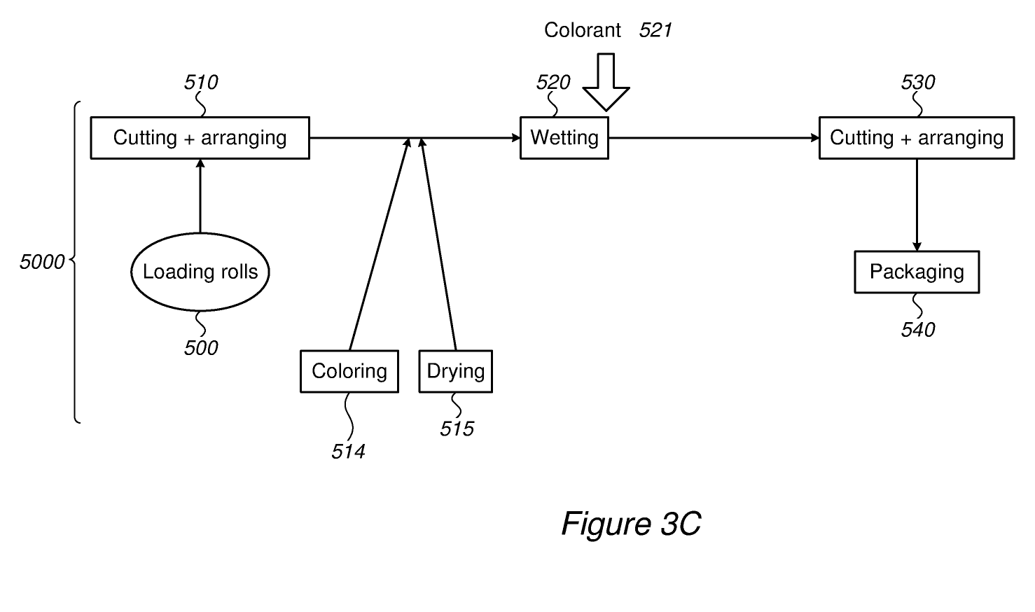

FIG. 3A illustrates, in more detail, the spunlace process (also called Hydroentanglement) 93 of creating nonwovens. The first step involves receiving compressed fibers 30 and unraveling them 300A-C. Most nonwovens are comprised of at least two or three types of fibers. The types of fibers are chosen according to the desired properties of the nonwovens, such as strength, elasticity, color etc. Sometimes nonwovens are comprised of only one type of fiber. A carding process is applied. Each bale (compressed) type of fiber is inserted to a separate machine unit to preliminary unravel the fibers and weigh them. Elements 300A-C each represent an unraveling machine unit. There may be more or less unraveling units depending on number of different types of fiber used. All the unraveled fibers are fed into a fiber mixing machine unit 310 (92) or alternatively placed on a conveyer belt on which the fibers start mixing and continue unraveling. Following the fiber mixing the nonwovens are brushed and assembled in desired patterns on a conveyer belt(s). The conveyer belt(s) inserts the fibers in to a machine with high speed water jets 320 which cause fiber entanglement thus creating the nonwovens. The nonwovens are then dried 330; usually they are first de-watered and then dried in an oven or through hot air drums. The dried nonwovens are then winded up 340 to produce nonwoven fabric rolls. The rolls are then placed in storage (70).

FIG. 3B illustrates an example of arranging steps 96 in which the fabric rolls are interfolded 380 (represented by the zig zags in the balloon). In some embodiments, the fabric may be cut into the desired wet wipe size and then interfolded 380 and wetted 390 (97). In some embodiments, the fabric may be slit into an intermediate width and then interfolded 380 and wetted 390; the cutting to the desired wet wipe size may be done after the wetting 390 stage.

Once the process of producing nonwoven fabric rolls in a certain color is complete in order to switch to a different color (or no color, such as white or other natural colors) all the machinery, described above, i.e. the unraveling machine units, the mixing machine unit, the conveyer belt, the high speed water jets, the oven or hot air drums etc. must be cleaned so no colored fiber residues are left. Cleaning the machinery is very time consuming and, in some cases, can take up to 12 hours. After the intensive cleaning a test run must be completed in order to verify that no color fibers are left in the system. In addition, the machines from the converting process also need to be cleaned between different colored nonwoven fabric rolls.

Accordingly, changing color according to the prior art requires intensive cleaning and wasting fibers on test runs. Because of these disadvantages manufactures make large batches of each color in order to avoid the extensive cleaning required for a color change.

Moreover, this process may not be an efficient method when small runs are needed. Coloring the fibers for the production of small runs can be costly and some colorants may not be able to be applied to the fibers prior to forming the fabric, or they may not perform well when applied to the fibers before the fabric assembly. Therefore, alternative processes for coloring wet wipes are needed which provide for fast, efficient, environmentally friendly and low-cost production.

The present invention provides novel coloring methods and systems.

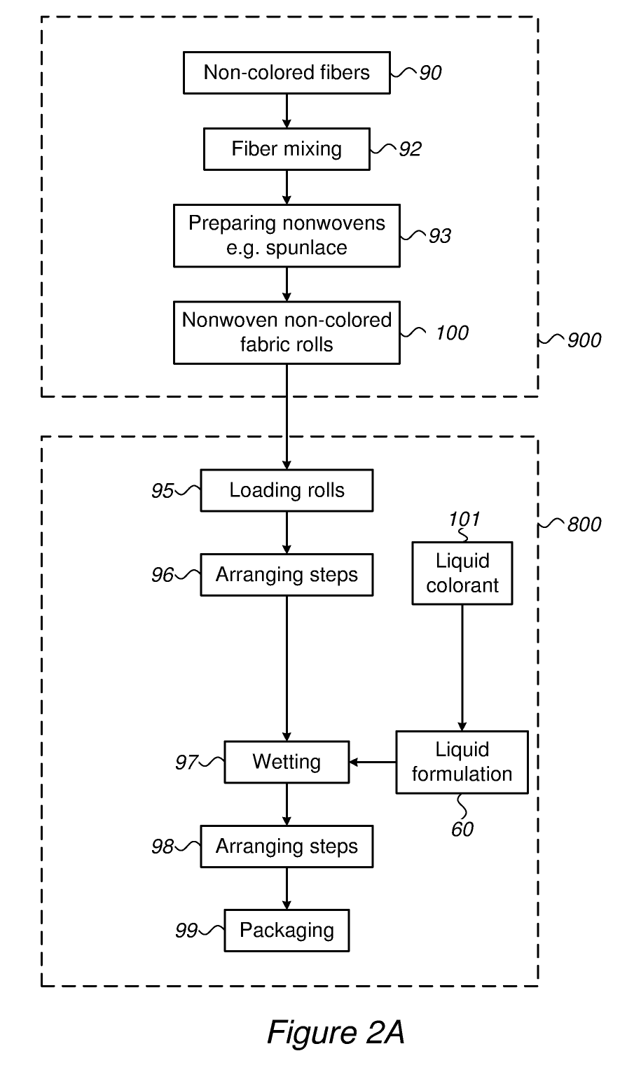

FIGS. 2A and 2B illustrate embodiments of the invention in which the coloring is preformed in the second section 800 of preparing wet wipes as apposed to the prior art, illustrated in FIG. 1, in which the coloring is preformed in the first section 900 of creating the nonwoven fabric. In FIGS. 2A and 2B the first section 900 illustrates unraveling one or more types of non-colored fibers 90 (such as white or other natural fiber colors) followed by mixing 92 and nonwoven preparation 93, such as the spunlace method, to produce non-colored nonwoven fabric rolls 100.

For the second stage 800 the non-colored fabric rolls 100 are loaded to a machine unit 95 and arranged 96. The arranging steps may include cutting the fabric into the desired wet wipe width or slitting to an intermediate width. The arranging steps may include, in addition or instead of the cutting/slitting, folding the fabric, such as the interfolding described in FIG. 3B, or other arranging steps such as stacking, layering, pressing etc.

FIG. 2A illustrates one embodiment of the invention in which, following the arranging steps 96, the fabric is wetted 97. Wherein the wetting is performed with a liquid colorant formulation (more details below) 101 and a liquid formulation 60 comprising a functional liquid. In some embodiments liquid formulation 60 is the same as liquid formulation 80.

FIG. 2B illustrates one embodiment of the invention in which, following the arranging steps 96, the fabric is colored 101 with a liquid colorant formulation. The color fabric is then wetted 97 with liquid formulation 60 comprising a functional liquid.

In some embodiments the fabric is dried 103 following the coloring 101 and prior to the wetting 97.

FIGS. 2A and 2B further illustrated passing the wet colored fabric through additional arranging steps 98 which may include cutting to the desired wet wipe size if that was not done in the first set of arranging steps 96. The arranging steps may include, in addition or instead of the cutting, folding the fabric, such as interfolding described in FIG. 3B, or other arranging steps such as stacking, layering, pressing etc. The wet wipes are then packaged 99. The wipes may be packed one by one (sachets), i.e. each wipe separately, such as used in restaurants and airlines, or multiple wipes in a package (flowpack), such as used for baby wipes or cleaning wipes.

In another embodiment, individual wet wipes may be packaged in sealed pouches (such as foil pouches). In this process, sheets of the packaging material are folded into small pouches and sealed from three sides to form open envelopes. Simultaneously, another conveyor line feeds a single non-woven colored wipe after it was colored in step 101 and dried in step 103 and prior to the wetting 97. The wipes are then wetted 97 directly in the open envelope and immediately after that the pouch is sealed.

Coloring the nonwovens means coloring substantially all of the fibers in the nonwoven wipe.

One of the advantages of producing the colored wet wipes of the current invention is the flexibility of color change. Manufactures can easily change the color of the wipes at any time and make small batches. For instance, producing limited editions such as making a special color for a holiday (such as orange for Halloween and red for Christmas) or for a specific client. Other non-limiting examples may be a series of wipes for cosmetics, such as make-up removers, each wipe having a different smell connected to the color of the wipe or ascribed to an ingredient used in the formulation (e.g. purple for beetroot), each color can also mark a different type of make-up remover such as pink for lipstick and yellow for eye shadow.

Coloring the fabric itself does not require working with colored fibers. Small quantities of colored fabrics can be produced by coloring with various colorants. The processes described above are processes that are easier for production of colored fabrics in comparison to the existing methods in the market and with lower production costs.

In addition, the added colorant can have added value for the user. For example, adding β-Carotene which serves as an orange colorant and also as a skin conditioning agent which may help promote, repair and maintain the skin and may provide potent antioxidant protection, protecting the skin from wrinkles and premature aging. Another example is adding Turmeric, which serves as a yellow colorant and also as a skin condition agent which may have anti-inflammatory and antioxidant properties.

Advantageously, with respect to prior art using colored fibers, disclosed embodiments enable production of small batches of colored wipes and do not require extensive cleaning of the whole production line from colored fibers when switching to production of uncolored wipes, or wipes colored with a different color. The prior art requirements for cleaning the production line are typically stringent as no traces of colored fibers are allowed and as the machinery and containers involved are very large, and the fibers are very fine. In contrast, disclosed embodiments add colorants only at the converting stage, and therefore do not affect the whole production line. While color stability may be lower than using pre-colored fibers, disclosed color compounds provide sufficient adherence to the wipes to efficiently color them without risk of color smearing on the user.

Advantageously, with respect to prior art using colored paper and/or colored tissues, it is emphasized that disclosed embodiments provide wet wipes, which are much more challenging to color than dry paper/paper tissues. While coloring and printing technologies for dry paper are well known, wetting is generally not applied to packed paper and or paper tissues due to their low structural integrity in wet conditions. On the other hand, known paper printing technologies are generally not applicable to wet wipes due to the different material structure, consistency and wetting of the wipes, which render paper-related techniques not applicable to wet wipes and accordingly a person skilled in the art would not have modified the wetting process of wet wipes to introduce color compounds.

FIG. 3C illustrates an apparatus 5000 which may be used for the converting process of section 800 illustrated in FIGS. 2A and 2B. Initially, non-woven material rolls are loaded to the apparatus 500, the fabric is then cut into the final wipe size or slit to an intermediate width and arranged in different ways 510 such as in piles of the desired number of wipes per package or interfolded such as in FIG. 3B. The fabric is then wetted 520 with a liquid formulation comprising a functional liquid, in some embodiments the liquid formulation includes a colorant 521. Following the wetting the fabric is then cut into the final size (if not previously done in step 510) and arranged to the desired amount in each package 530. The colored wipes are then packaged 540. In some embodiments the fabric is colored 514 with a liquid formulation comprising a colorant, between the initial cutting/slitting and arranging step 510 and the wetting stage 520 and in this embodiment the wetting stage does not include colorant 521. Further, in some embodiments, following the coloring step 514 the fabric is dried 515 and only then proceeds to the wetting stage 520.

In one embodiment, this invention provides an apparatus (such as 5000 in FIG. 3C) for producing a colored wet wipe, comprising: a fabric conveyor belt; a converting unit comprising a liquid application unit (520); wherein said application unit comprises a source of colorant (a reservoir). The apparatus may further comprise a packaging unit (540).

In one embodiment, the liquid application unit (520) comprises a nozzle(s) for applying the liquid formulation and the liquid colorant formulation onto the fabric and a reservoir containing the liquid colorant formulation. The apparatus may further comprise a heater, a temperature regulator, a temperature gauge, a humidity gauge, a pressure gauge or a combination thereof.

The apparatus may further comprise a press or other surface treatment unit (such as 510 and/or 530).

In one embodiment, this invention provides an apparatus for producing a colored wet wipe, said apparatus comprising: a fabric conveyor belt; a converting unit comprising:

a first liquid application unit (514); and a second liquid application unit (520); wherein said first application unit comprises a first liquid, said liquid comprising a colorant (liquid colorant formulation), and wherein said second application unit comprises a second liquid, said second liquid does not comprise the colorant (liquid formulation).

In one embodiment, the apparatus further comprises a drying unit (515), the drying unit is located between the first and the second application units.

In one embodiment, the apparatus further comprises a packaging unit (540).

In one embodiment, the first liquid application unit, the second liquid application unit or a combination thereof comprise a nozzle(s) for introducing the liquid(s) onto the fabric.

In one embodiment, the apparatus further comprises a heater, a temperature regulator, a temperature gauge, a humidity gauge, a pressure gauge or a combination thereof. In one embodiment, the apparatus further comprises a press.

In one embodiment, any colorant described herein for processes of this invention can be included in the liquids contained and used in the application units of apparatuses of this invention. Similarly, any liquid or liquid constituent described herein above for processes of this invention can be included as or in the liquids contained and used in application units of apparatuses of this invention.

The application units described herein above are used to apply liquid to the fabric. Application of liquid to the fabric can be conducted in any manner known in the art. For example, applying a liquid to the fabric may be achieved by spraying, sprinkling, dripping or drizzling the liquid onto said fabric, or wetting, dipping, soaking, spotting or immersing said fabric in/by said liquid, or printing said liquid on said fabric. Application of liquid to the fabric involves ultrasound in some embodiments. According to this aspect and in some embodiments, the fabric is dipped or soaked in a liquid and ultrasound is applied to the liquid and to the fabric. The ultra sound energy facilitates/strengthen bonding of colorant to the fabric in some embodiments. In some embodiments, ultrasound energy assists the process of colorant-penetration into the fabric.

Applying a liquid to the fabric may be achieved by rolling said fabric between rollers, the rollers comprising the liquid and/or having the liquid on their surface. The application units can be modified to be compatible with any application method described herein above. Accordingly, in some embodiments, the application units comprise one or more of the following: nozzles, roller(s), containers, hoses, pressurized containers, levers, handles, pedals, press, knobs, injectors, pumps, valves, printing devices etc. The application units may further comprise a heater, a cooling device, a temperature regulator, a temperature gauge, a humidity gauge, a press, a pressure gauge a weight-measuring device, a volume-measuring device, a density measuring device or a combination thereof. The application units may be controlled by a computerized system and may be automated in some embodiments. The elements described herein for the application units may be included in other portions of the apparatus in some embodiments. Portions of the apparatus may be operated manually or automatically as needed. The apparatuses include or are connected to a fabric feed line in some embodiments.

In some embodiments, following the coloring process, the wipe is packed. The wipe(s) are packed in a packaging unit or on a packaging line in some embodiments.

### The Fabric

A “wet wipe” is fibrous web (also called “fabric”), including paper, which may be woven, knitted or non-woven. A “wet wipe” is capable of being wetted by a liquid with a wetting ratio in a range of 0.5-1, 1-5, 1-10, 1-20, 1-30, 1-50, 1-75, 1-80, 0.1-10, 10-20 or 10-50 or 1-100 and packaged in wet form.

In general, methods of this invention may be applicable to any fabric weight used for a wet wipe product. In one embodiment, the density of the fabric prior to the wetting step(s) is up to 400 grams per square meter (“gsm”), between 10-300 gsm, between 20-300 gsm, between 1-300 gsm, between 0.5-50 gsm or between 0.1-100 gsm. In one embodiment, the shape of the (unfolded) wipe is a square, a rectangle or a strip. In other embodiments the wipe may assume any shape. In some embodiments the wipes are packaged in packs of two or more wipes. In other embodiments, the wipes are individually packed.

In one embodiment, the fabric is a non-woven fabric.

All non-woven fabrics, e.g. 100% viscose, polyester, rayon, polypropylene, different ratios of viscose-polyester, cotton, polyethylene, etc., can be colored and used in the methods and systems described herein. In addition, to be used are also fabrics with different surface finishes (anionic, cationic, hydrophilic, hydrophobic, etc.) for wetting with different colorant based formulations according to this invention.

In one embodiment, the fabric comprises or consists of polyester, viscose, rayon, cotton, polypropylene or polyethylene, or any combination thereof. In one embodiment, the fabric comprises viscose and polyester. In one embodiment, the viscose to polyester percentage fiber ratio in the fabric ranges between 0.001%/99.999% to 99.999%/0.001%. In one embodiment, the fabric comprises cellulose. In one embodiment, the fabric comprises cellulose fibers. In one embodiment, the fabric comprises pulp. In one embodiment, the fabric is or comprises paper. In one embodiment, the fabric is or comprises cloth. Any other synthetic, artificial, natural, or nature-derived fabrics such as canvas, nylon, silk, flax, processed cellulose, and the like can be used as fabrics in processes of this invention.

Fabric that has various different surface properties can be obtained either by using sieves with the required finish properties or by applying physical and/or chemical treatments to the fabric.

In some embodiments, the surface of said fabric comprises a finish. The finish may facilitate colorant adhesion to the fabric. In one embodiment, the finish comprises an anionic finish, a cationic finish, a hydrophilic finish or a hydrophobic finish or a combination thereof. In one embodiment, a fabric comprising a finish is a treated fabric. In one embodiment, the finish is designed in view of the colorant. The finish may be suitable for adherence of a certain colorant.

In some embodiments, the fabric may be treated chemically or physically. For example, the surface of the fabric can be treated physically by applying pressure, reducing pressure, changing the temperature, or by applying mechanical force such as brushing, polishing, rubbing, wetting, drying or airing, gassing, or degassing the fabric.

In some embodiments, the surface of the fabric may be treated chemically to modify the chemical groups on the surface of the fabric. For example, chemical treatment can form or eliminate, reduce or increase the number of hydroxyl, carboxyl, nitro, fluoro, amino groups or any other chemical group that defines the surface properties. The surface properties exhibited by the surface chemical groups affect adhesion of colorants to the surface. In some embodiments, chemical treatment of the fabric results in one or more of anionic, cationic, hydrophobic or hydrophilic surface.

### Color and Colorant

The wipes color is controlled by the percent of the colorant in the liquid formulation. A desired color can also be achieved by mixing several pigments/dyes/colorants at a defined percentage in the formulation. The percentage of the colorant(s) in the liquid formulation can be adjusted according to the desired color.

The desired colorant is mixed homogenously in the carrying liquid. In some embodiments the carrying liquid and the colorant homogenously wet and color the fabric. In some embodiments at least, half of the colorant attaches to the fabric.

The desired colorant mixes homogenously with the carrying liquid and/or all the components of the liquid formulation and wets the nonwoven fabric homogenously. The desired colorant does not come off when used. For example, if used to wipe hands does not come off on the hands or if used to wipe a table does not come off on the table or on the user's hand. The desired colorant does not fade quickly and does not fade unevenly.

In some embodiments, the colorant comprises a mixture of two or more colorants.

In one embodiment, the colorant is or comprises a dye, a pigment or a combination thereof. In one embodiment, the colorant is a natural color, a nature-derived color, a nature-identical color or an artificial color or any combination thereof. In one embodiment, the colorant is a liquid formulation comprising a natural colorant. The natural colorant may be a nature-derived color, a nature-identical color, or any combination thereof. In one embodiment, the colorant comprises carotenoid such as β-carotene or lycopene, curcumin, annatto, red cabbage extract, caramel, beet juice extract, paprika, lycopene, carmine or chlorophylls or any combination thereof. In one embodiment, the colorant is made from or is an extract of a vegetable, an animal, a fungus, an insect, bacteria, a mineral, a plant, or algae or a combination thereof. In one embodiment, the color of said colored wet wipe is white, yellow, orange, pink, red, blue, green, purple, brown or black, or any of their corresponding hues or shades. In one embodiment, the colorant is a food colorant.

In one embodiment, the colorant comprises or consist of colored edible protein(s), colored oil, oil-based colorant or a mix of colorants. In one embodiment, the colored oil is an essential oil.

In one embodiment, the natural colors are environmentally-friendly. In one embodiment, the natural colors are suitable for cosmetic applications of the wipe as they do not pose any health concern in contrast to some synthetic colors.

In one embodiment, the natural color is a natural color obtained by a synthetic process, defined as a “nature-identical” color. In some embodiment, the natural color is extracted from a natural source, and further undergoes additional synthetic treatment.

In one embodiment, the colorant is a natural dye, a synthetic dye or a combination thereof. In one embodiment, the synthetic dye comprises a pH basic dye, an acid dye, a direct dye, a disperse dye, a reactive dye or a pigment dye or any combination thereof.

The liquid colorant formulation comprising one or more natural or nature identical colorants may further include: water, an aqueous solution, an organic solvent, oil, water-based extracts, extract, leaf extract, leaf water or flower water or any combination thereof.

In one embodiment, the liquid colorant formulation comprises water, and the water content in the liquid colorant formulation ranges between 80%-99.9999%, between 85%-99.9999%, between 95%-99.9999% or between 50%-99.9999% (wt %). In one embodiment, the liquid colorant formulation comprises water, and the water content in said liquid colorant formulation is more than 85%, more than 95% or is between 80%-99% (wt %). In one embodiment, the water content is between 0.0001% and 99.9999% of the total liquid colorant formulation weight.

In other embodiments, the liquid colorant formulation comprise oil. In one embodiment, the liquid is an organic solvent.

In embodiments, the liquid carrier may comprise 100% water, 100% oil, or 100% organic solvent.

In one embodiment, the wetting ratio of the liquid colorant formulation with respect to said wipe ranges between 0.1-0.5, 0.5-1, 1-5, 1-10, 1-20, 1-30, 1-50, 1-75, 1-80, 0.1-10, 10-20 or 10-50 or 1-100, wherein said wetting ratio is measured as liquid weight divided by wipe (fabric) weight. In one embodiment, the wetting ratio of said liquid with respect to said wipe ranges between 0.001-1, 0.01-1 or 0.1-1. In one embodiment, the wetting ratio of the liquid with respect to the wipe is 2, 3 or 20 (weight of liquid divided by weight of fabric).

Adhesion or bonding or fixation of the colorant to the fabric can be achieved by different mechanisms. For example, the colorant may be intercalated within the fabric. The colorant may be held in pores and voids within the fabric or on the fabric's surface. The colorant may be trapped in voids/pores between the sieves, in voids/pores within the sieves or in both. The colorant may be fixed in the fabric due to entanglement and entrapping in the fabric or entanglement and entrapping on the fabric's surface.

In some embodiments, the adherence of the colorant to the fabric involves chemical bonds or chemical forces. The colorant can be adhered to or bonded to the fabric through covalent bonds, hydrogen bonds, polar bonds, van der Walls forces, electrostatic interactions, etc. The adhesion of the colorant to the fabric may be based on hydrophobic-hydrophobic interactions between hydrophobic colorant and hydrophobic fabric, hydrophilic-hydrophilic interactions between hydrophilic colorant and fabric, cationic-anionic interactions between anionic fabric and cationic colorant or vice versa etc. Polar or non-polar interactions between colorant and fabric etc. Fabric surface modification may be performed by forming a self-assembled monolayer or self-assembled multilayers on the surface of the fabric.

In some embodiments successful adherence of the colorant is defined by squeezing the colored wet wipe and having no colorant in the extracted liquid.

It is to be noted that in some embodiments, and in some colorant formulations, the colorant is encapsulated in a matrix, or is attached or enclosed or partially covered by another material such as a polymer or any other protective layer. For example, the polymer may be acrylic polymer. According to this aspect, the interactions described above apply to the interactions between the fabric surface and the material that is associated with the colorant. For example, if the colorant is in the form of a particle covered by a shell, the interaction of the shell and the fabric will govern adhesion of the colorant to the fabric. The interaction between such shell/layer and the fabric can be any of the interactions described herein above between the colorant and the fabric.

Binders may be incorporated in formulations comprising colorants and are materials that bind the colorant. Non-limiting examples of binders are Acrylates octylacrylamide copolymer, Ammonium Acrylates Copolymer and acrylates/dimethylaminoethyl methacrylate copolymer. In some embodiments, the binder facilitates adhesion of the colorant to the fabric. In one embodiment, the binder coats the colorant and keeps it intact. In one embodiment, the binder is the material that adheres to the fabric and the colorant is trapped within the binder. In some embodiments, the binder is a polymer. In some embodiments, the binder is an organic polymer. In some embodiments, the binder is used to encapsulate the colorant. In some embodiments the encapsulated colorant is a pigment. Pigments can be encapsulated in certain binders and maintain their colors. Binders stabilizes the colorants in some embodiments. In some embodiments, the liquid applied to the fabric comprises the binder and a colorant. Following application to the fabric and in some embodiments, the fabric is dried. Drying the fabric solidifies or jellifies the binder, such that it forms a solid or gel layer of binder on the fabric. This solid/gel layer comprises the colorant. This layer fixes the colorant to the fabric. This binder layer stabilizes the colorant and keeps it from dissolving in the functional liquid or in any other liquid with which it makes contact. Using a binder enables or facilitates coloring of the fabric by colorants that do not adhere well to a certain fabric or that are not stable when applied to the fabric with no binder.

A chemical linker may be attached to colorant molecules or to colorant particles and this linker provides the attachment of the colorant to the fabric's surface. According to this aspect and in one embodiment, the linker is attached to the fabric's surface according to interactions as described herein above. In non-limiting examples the chemical linker may be: dihydroxy ethylene urea (DHEU), urea Formaldehyde, Melamine Formaldehyde, Monochlorotriazine, vinyl sulphone, HomoBifunctional and Heterobifunctional linkers.

Some of the above interactions between colorant and fabric can be described similarly with respect to the fibers, i.e. interactions between the colorant and the fibers of which the fabric is made.

As noted above, the colorant may be attached to another material (polymer matrix, particle shell, chemical linker etc.). The interaction between such materials and the fabric mediates the attachment of the colorant to the fabric. According to this aspect, the colorant is not directly attached to the fabric, it is attached through the other material. Any other process of binding the colorant to the fabric as known in the art is included in embodiments of this invention.

Coloring the nonwovens means coloring substantially all of the fibers in the nonwoven wipe.

In some embodiments, the wipes are disposable. According to this aspect, the wipes are used only once or a few times and are discarded. Accordingly, the requirements for the colorant and for the colored wipe are not as restrictive as compared to other colored fabrics. These characteristics enable use of colorants that are not stable for a prolonged time. These characteristics enable coloring the fabric as disclosed herein, as opposed to conventional techniques where each individual fiber is first colored, and the fabric is assembled out of colored fibers. For example, in embodiments of this invention, natural (or natural-derived/nature-identical) colors are used. Such colors are not used in the coloring of other fabrics/papers because they tend to degrade with time. The color of such colored goods starts to fade and after some time it may disappear completely. This effect may be accelerated when the colored fabric is exposed to light in some instances. According to embodiments of the invention, the wet wipes are only used for a short period of time and then disposed, and prior to use, may be kept in a package shielded from light.

The novel colors and color formulations may have added value when used in cosmetic applications in some embodiments (such as the β-Carotene and Turmeric described above). The novel colors/color formulations are environmentally friendly and do not present health or environment concerns in some embodiments.

Some examples of colorants are:

BioColor Red: which can adhere to the nonwoven fabric without any treatment to the fabric but chemical treatment to the fabric improves its adherence.
Acid orange II, E-100 yellow sunset and BioColor Blue all which adhered to chemically treated nonwoven fabric.
BioColor Yellow did not adhere to non treated nonwoven fabric and caused non desired chemical reactions when used with chemically treated nonwoven fabric.

### Liquid

A “wet wipe functional liquid” is a liquid that imparts wet wipe functionality to a wipe so that the fabric of the wipe is wet when taken out of the package, for example to wipe the skin (personal use) or a household surface (household use). Generally, a wet wipe functional liquid comprises at least water or an organic solvent and a functional formulation. Additional or other functional ingredients may be added to the wet wipe functional liquid.

“Other functional ingredients” may include a humectant, a detergent, a surfactant, a preservative, a skin conditioning agent, an extract, a skin-care material, a solubilizer, a skin protectant, a cleaning agent, an emollient or a perfume or any combination thereof that adds functionality to a wet wipe product.

“Functional formulation” is a formulation that is directed to a certain use. Functional formulations include but are not limited to cosmetic formulations and household formulations. Cosmetic formulations may be directed to one or more of cleaning, skin treatment, moisturizing, nourishing, exfoliating, toning, pampering, cooling, peeling, oiling, reviving, applying a lotion, removing make-up, removing other materials/formulations, applying a pharmaceutical formulation, applying an active material, formulations used for fragrance etc. Household formulations include but are not limited to cleaning formulations, formulations for removing materials, applying materials such as lubricants/waxes, brighteners, applying materials for polish, gloss, glow, shine, formulations used for etching, peeling, polishing, formulations used for fragrance and the like. In some embodiments, functional formulations consist of a solvent (one or more solvents), for example, a formulation may be water only or ethanol only, and such formulation can provide one of the functions described above (e.g. cleaning).

“Personal care” in the context of this invention refers to cosmetics in one embodiment. In another embodiment, personal care refers to beauty products and hygiene products. In addition, cosmetic and personal care wet wipes of this invention also relate to therapeutic wipes and to therapeutic formulations contained in the wet wipes. In this context and as described above, the wet wipes may contain a drug, a pharmaceutical formulation, a therapeutic ingredient, a disinfectant, water or wound cleaning liquid. The wipe may be used to simply wipe an area, or it can be kept on that area (e.g. a skin area) for an extended period, for example as a mask or as a bandage.

“Household formulation” include the formulations described herein above and any formulation used for cleaning or for other related processes. For example, household formulation refers also to formulations used for all purpose cleaning, for lamination, for polishing, brightening, applying color to items such as furniture, floors, cars and bicycles, arts and crafts items, silverware, etc. In one embodiment, colored wet wipes of this invention are used for the purposes described herein above in construction projects, for beautification, for renovation, for remodeling, in the production of decorative items and any other action taken for such purposes at home, in the yard or garden, or as applied for cars and bikes etc. It is to be noted that the uses described above are provided as examples only. Wet wipes of this invention may be used for many other applications in many different areas. For example, wet wipes of this invention may be used in hospitals, in clinics and in care centers, in offices, in factories, in auto-repair centers, for military uses etc.

In one embodiment, the liquid formulation comprises a colorant and a functional liquid.

In one embodiment, the liquid containing colorant, the wet wipe functional liquid, or a combination thereof comprise a carrier. The carrier may comprise or consist of: water, an aqueous solution, an organic solvent, oil, water-based extracts, extract, leaf extract, leaf water or flower water or any combination thereof.

In one embodiment, the liquid containing colorant, the wet wipe functional liquid, or a combination thereof comprise: a humectant, a detergent a surfactant, a preservative, a skin conditioning agent, an extract, a skin-care material, a solubilizer, a skin protectant, a cleaning agent, an emollient or a perfume or any combination thereof.

Below is an example of a composition of the liquid formulation:

### Liquid Formulation I:

| Description | Quantity (%) |
|---|---|
| Annatto | 0.05-1.0 |
| β-Carotene | 0.05-1.0 |
| Perfume | 0.07-0.5 |
| Vitamin E | 0.001-0.1 |
| Daucus Carota Sativa Ext | 0.01-0.5 |
| Phenoxyethanol | 0.3-0.999 |
| Polyglyceryl-4 Caprate | 0.3-1.5 |
| Xanthan Gum | 0.01-1.5 |
| Water-Pipeline material-Liter | completion to 100% |
| Sodium Benzoate | 0.05-4.9 |
| Benzoic acid | 0.04-0.2 |
| Caprylyl/Capryl Glucoside | 0.3-1.5 |
| Dehydroxyacetic acid | 0.02-0.2 |
| Glycerin | 0.05-5 |
| Citric Acid | 0.005-0.05 |

Where the colorant is in a liquid formulation, the colorant may be present in said liquid in a range of 0.0% to 80% (wt %); 0.0% to 99% (wt %). In one embodiment, the colorant percentage in said liquid ranges between 0.0% to 10% (wt %).

In some embodiments, the colorant is dissolved or dispersed or suspended in said liquid.

In some embodiments, a binder is added to the colorant liquid to bind the colorant in the liquid.

In one embodiment, applying a first liquid to said fabric comprises one or more of the following: spraying, sprinkling, dripping or drizzling said liquid onto said fabric, or wetting, dipping, soaking, spotting or immersing said fabric in/by said liquid, or printing said liquid on said fabric, or rolling said fabric between rollers wetted by said liquid.

In one embodiment, processes of this invention comprise additional steps applied to the wipes including but not limited to wipe lamination, texture manipulation, smoothing, pressing, cutting, folding, partial drying, packing, or any combination thereof.

A fluid comprising a colorant can be kept as a liquid under pressure. When applied to the fabric, the liquid can be ejected from a pressurized container in the form of an aerosol comprising the colorant. The colorant adheres to the fabric and the liquid turns into gas and disappears or disperses in the surroundings. In some embodiments, the fluid is a gas carrying the colorant, the gas is kept in a closed reservoir and is applied to the fabric when needed through hoses, nozzles, or the like. The colorant adheres to the fabric and the gas disperses in the surroundings. CO₂ is one example of a fluid that can be used in such embodiments.

Following applying a colorant with the wet wipe functional liquid or following applying said wet wipe functional liquid after applying the colorant, the wipe is generally packed in a closed package to maintain a moist or wetted state. In some embodiments, before packaging, the fabric undergoes other cutting and/or folding, or any other desired pre-packaging process.

### Methods

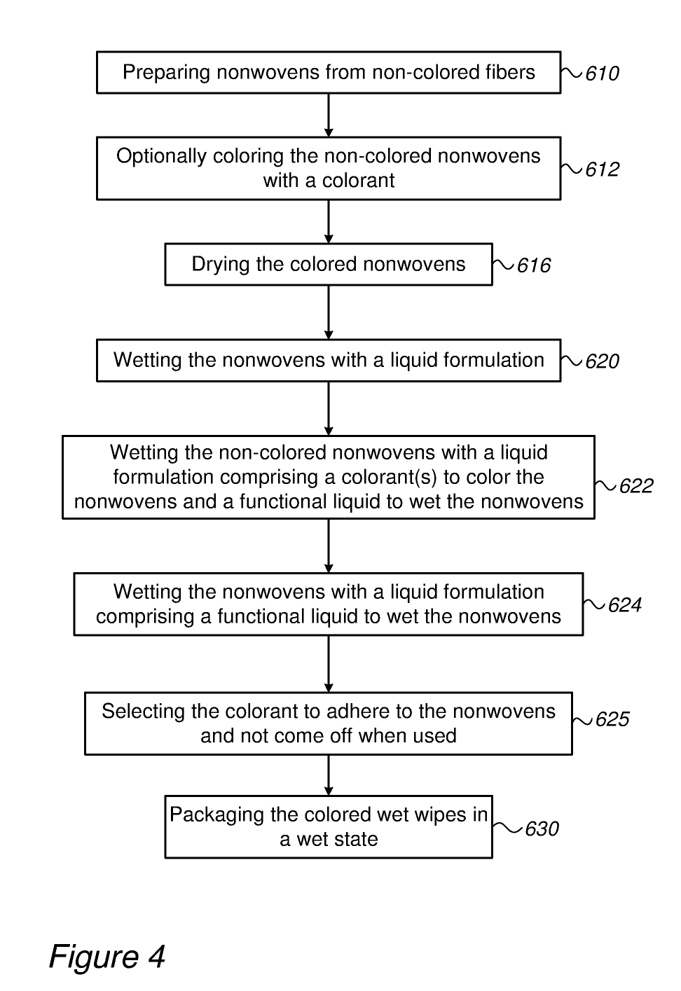

FIG. 4 describes a method of preparing colored wet wipes according to embodiments of the invention. In one embodiment the method comprises preparing nonwovens from non-colored fibers 610, wetting the non-colored nonwovens with a liquid formulation 620. In one embodiment the liquid formulation comprises a colorant to color the nonwovens and a functional liquid to wet the nonwovens 622. In one embodiment the liquid formulation comprises a functional liquid 624. In one embodiment the non-colored nonwovens are colored with a colorant 612 prior to the wetting with the functional liquid. In one embodiment the colored fibers are dried 616 prior to the wetting with the functional liquid formulation. In one embodiment the colorant is selected to adhere to the nonwovens and not come off when used 625. In one embodiment the colored wet wipes are then packaged in a wet state 630.

### EXAMPLES

### Example 1

### One-Step Wet and Color Process

Wet wipes were produced as follows: a non-colored fabric was fed to a production line. The fabric passed an application unit comprising nozzles. The nozzles were connected to a liquid reservoir. The liquid reservoir contained a liquid colorant formulation (Liquid formulation I detailed above with the colorants Annatto and β-Carotene or the colorants listed below). The formulation comprising the colorant was applied to the fabric through the nozzles. The fabric was colored by the colorant in the liquid. The fabric was wetted by the liquid. The resultant was a wet colored fabric (a wet colored wipe). The wet colored wipe was packaged in a closed package.

In different trials, the colorants used were as follows: Orange color: mixture of Napture Col orange LC217 and Napture Col Orange LC 214L, Pink or red color: Napture Col red LC 313.

### Example 2

### Two-Step Color and Wet Process

Wet wipes were produced as follows: a non-colored fabric was fed to a production line. The fabric passed an application unit comprising nozzles. The nozzles were connected to a liquid reservoir. The liquid reservoir comprised a liquid comprising a colorant. The liquid comprising the colorant (Annatto and β-Carotene or the colorants listed below) was applied to the fabric through the nozzles. The fabric was colored by the colorant in the liquid. The fabric was wetted by the liquid.

Subsequently, the fabric passes through a drying unit, such that the fabric was dried, but the colorant remained adhered to the fabric.

The dry fabric was passed through a second liquid application unit, this unit comprised the functional cosmetic/house hold liquid but no colorant (Liquid formulation I, detailed above, without the colorants Annatto and β-Carotene). This liquid was applied to the fabric through nozzles. The resultant was a wet colored wipe. The wet colored wipe was packaged in a closed package.

In different trials, the colorants used are as follows:

Orange color: mixture of Napture Col orange LC217 and Napture Col Orange LC 214L; Pink or red color: Napture Col red LC 313.

1. A system for manufacturing colored wet wipes, the system comprising:
a preparing unit configured to prepare nonwovens from non-colored fibers;
at least a first liquid supply reservoir;
at least one wetting unit configured to apply at least a first liquid formulation from the first liquid supply reservoir to the nonwovens; and
a packaging unit adapted to wrap the wet wipe in a package in a wet state;
wherein the first liquid formulation comprises a colorant; and wherein a functional liquid adapted to wet the nonwovens and maintain the colored wet wipes wet in the package is included in the first liquid formulation or applied separately.

2. The system of claim 1, wherein the at least one wetting unit comprises at least one first sub-unit configured to apply a liquid colorant in the first liquid formulation and color the nonwovens with the liquid colorant to produce colored nonwovens, and a least one second sub-unit configured to apply a second liquid formulation comprising a functional liquid and wet the colored nonwovens with the functional liquid to produce colored wet wipes.

3. The system of claim 2, further comprising a drier configured to dry the colored nonwovens intermediate the first sub-unit applying the liquid colorant and the second sub-unit wetting with the functional liquid.

4. The system of claim 1, wherein the preparing unit is a spunlace unit configured to prepare spunlace nonwovens by hydroentanglement.

5. The system of claim 1, wherein the first liquid formulation comprises a colorant and a functional liquid.

6. A method for manufacturing colored wet wipes, the method comprising:
preparing nonwovens from non-colored fibers;
wetting the non-colored nonwovens with a liquid formulation comprising a colorant to form colored nonwovens;
wetting the colored nonwovens with a functional liquid adapted to maintain the wet wipes in a wet state to form colored wet wipes; and
packaging the colored wet wipes in a wet state.

7. The method of claim 6, comprising wetting the non-colored nonwoven with the colorant and the functional liquid simultaneously in a single wetting step.

8. The method of claim 6, comprising wetting the non-colored nonwoven with the liquid composition comprising a colorant and thereafter wetting the colored nonwovens with the functional liquid to form a wet wipe.

9. The method of claim 8, further comprising: drying the colored nonwovens prior to wetting with the functional liquid.

10. The method of claim 6, comprising preparing the nonwovens by hydroentanglement to obtain spunlace nonwovens.

11. The method of claim 6, wherein the functional liquid comprises a water or an organic solvent and a functional ingredient for personal or household use of the wet wipe.

12. The method of claim 6, further comprising electing the colorant to adhere to the nonwovens and not come off when used.

13. Wet wipes colored with a colorant, wherein the colorant is configured to adhere to the nonwovens; and wherein the colorant comprises an extract of: a vegetable, an animal, a fungus, an insect, bacteria, a mineral, a plant, or algae or a combination thereof.
